(19) United States
(12) Patent Application Publication
(10) Pub. No.: US 2012/0111330 A1
(43) Pub. Date: May 10, 2012

(54) MASK FOR PROVIDING A VISUAL CUE

(76) Inventor: Kristina Ann Gartner, Winchester, MA (US)

(21) Appl. No.: 13/285,342

(22) Filed: Oct. 31, 2011

Related U.S. Application Data
(60) Provisional application No. 61/411,312, filed on Nov. 8, 2010.

(51) Int. Cl. A62B 9/00; A61M 16/06

(52) U.S. Cl. 128/205.23

(57) ABSTRACT

The invention provides, in part, visual indicators for respiratory masks, such as those used in hospital settings. The visual indicator is a mark that can be applied to or incorporated in a respiratory mask. The visual indicator changes color and/or opacity when the patient exhales and indicates whether or not the patient has respiratory exchange at any given point in time. The invention further provides systems and methods for monitoring patient respiratory exchange.

### REFERENCE TO RELATED APPLICATIONS

This application claims priority to and the benefit of U.S. Provisional Application No. 61/411,312, filed on Nov. 8, 2010, the entire contents of which are hereby incorporated by reference.

### FIELD OF THE INVENTION

The invention relates to a mask, e.g., a surgical respiration mask, which provides a visual cue to monitor the rhythmic exhalation process and respiratory pattern of a patient.

### BACKGROUND

Respiratory masks are well known in the medical field for administering oxygen, monitored anesthesia care (MAC), and post anesthesia care to patients. As the patient breathes into a respiratory mask, each exhaled breath has a moisture content of approximately 4% by volume of exhaled air. This is equivalent to a relative humidity value of 100%, which is the saturation limit for air. There is approximately 500 cc (ml) of air in every exhaled breath, and each exhaled breath has approximately 20 ml of water at body temperature and atmospheric pressure. In addition, exhaled air has an approximate temperature range of about 89 to about 101 degrees Fahrenheit.

### SUMMARY

The invention relates to visual indicators for respiratory masks, such as those used in hospital settings. The visual indicator is a mark that can be applied to or incorporated in a respiratory mask. The visual indicator changes color and/or opacity when the patient exhales, and the mark indicates whether or not the patient has respiratory exchange at any given point in time. In some embodiments, the visual indicator may change color in response to, for example, variations in temperature, moisture, CO₂ concentration, O₂ concentration, etc., that occur when a patient breathes into the mask. The visual indicator allows a patient care provider to quickly assess a patient's respiratory exchange without the need for complex medical equipment or care provider judgment. In addition, the visual indicator is well suited for monitoring patient breathing while a patient is in transit, such as in an ambulance, post anesthesia care, or between rooms in a hospital, because the visual indicator is easily observable and is self-contained. Respiratory masks incorporating visual indicators of the invention also can be inexpensive to manufacture and disposable.

The invention provides, in part, a respiratory mask having a body shaped to enclose a patient's nose and mouth. The mask is configured to deliver breathable gases to the patient. The respiratory mask includes a visual indicator associated with the body, and the visual indicator is capable of transitioning from a first color state to a second color state in response to a patient's respiratory exchange. In some embodiments, the visual indicator is a color changing dye. In some embodiments, the visual indicator transitions from the first color state to the second color state when the patient exhales. In some embodiments, the visual indicator is capable of transitioning from the second color state to the first color state between exhalations. In some embodiments, the visual indicator is responsive to temperature changes and can transition from the first color state to the second color state when the visual indicator reaches, for example, a temperature of about 89 degrees Fahrenheit. In some embodiments, the visual indicator is responsive to moisture contained in breath and/or to CO₂ levels contained in breath. In some embodiments, the first color state is selected from the group consisting of: clear, a visible color, opaque, translucent, and/or combinations thereof. In some embodiments, the second color state is selected from the group consisting of: clear, a visible color, opaque, translucent, and/or combinations thereof.

The invention also provides an indicator (e.g., a sticker, decal, or label) for monitoring patient respiratory exchange. The indicator includes a backing and a visual indicator disposed on the backing, and the visual indicator is capable of transitioning from a first color state to a second color state in response to a patient's respiratory exchange, and the indicator is adapted to be affixed to a respiratory mask. In some embodiments, the visual indicator is a color changing dye. In some embodiments, the visual indicator transitions from the first color state to the second color state when the patient exhales. In some embodiments, the visual indicator transitions from the second color state to the first color state between exhalations. In some embodiments, the visual indicator is responsive to temperature changes, and the visual indicator can transition from the first color state to the second color state when the visual indicator reaches, for example, a temperature of about 89 degrees Fahrenheit. In some embodiments, the visual indicator is responsive to moisture contained in breath and/or to CO₂ levels contained in breath. In some embodiments, the indicator includes an adhesive disposed on a first side of the backing, the adhesive capable of affixing the indicator to a respiratory mask. In some embodiments, the first color state is selected from the group consisting of: clear, a visible color, opaque, translucent, and/or combinations thereof. In some embodiments, the second color state is selected from the group consisting of: clear, a visible color, opaque, translucent, and/or combinations thereof.

The invention also provides a system for monitoring patient respiratory exchange. The system can include a respiratory mask and a visual indicator disposed on the respiratory mask. The respiratory mask is shaped to cover a patient's nose and mouth and configured to deliver breathable gases to the patient. The visual indicator is capable of transitioning from a first color state to a second color state in response to a patient's respiratory exchange. The system also can include an electronic monitor for monitoring the status of the visual indicator, and the electronic monitor can be configured to trigger an alarm if the visual indicator remains in the first color state for a period of time substantially longer than the duration of a single breath.

The invention also provides a method of monitoring patient respiratory exchange. In some embodiments, the method includes the steps of providing a respiratory mask having a visual indicator disposed on the respiratory mask, electronically monitoring the visual indicator, and electronically triggering an alarm if the visual indicator remains in the first color state for a period of time substantially longer than the duration of a single breath. Rhythmic transitions between the first color state and the second color state are indicative that the patient is exchanging normally.

### BRIEF DESCRIPTION OF DRAWINGS

The figures are not necessarily to scale, emphasis instead generally being placed upon illustrative principles. The figures are to be considered illustrative in all aspects and are not intended to limit the invention, the scope of which is defined by the claims.

FIG. 1 shows a prior art respiration mask.

FIGS. 2A-B show a respiration mask having a visual indicator, in accordance with an illustrative embodiment.

FIG. 3 shows a respiration mask having a visual indicator, in accordance with an illustrative embodiment.

FIG. 4 shows a respiration mask having a visual indicator, in accordance with an illustrative embodiment.

### DETAILED DESCRIPTION

The present invention includes a mask, such as surgical respiration mask. The mask is capable of fitting over a patient's nose and mouth, allowing for entrained air to enter the mask because the patient may need to breathe ambient room air along with oxygen from an oxygen source. The mask is preferably clear, and further preferably is made of plastic, silicone or vinyl. A transparent mask leaves the patient's face visible and allows health care providers to better ascertain the patient's condition. The mask can be lightweight and comfortably fitting on the patient's face to increase patient acceptance so as not to remove or pull it from the face when in place. The mask can also be made of a material that is disposable and can be biodegradable. The material used to construct the mask can be a clear, non-toxic PVC. The mask apparatus can further include an adjustable nose clip, which can be a bendable aluminum strip that fits over the patient's nose area, and non-crushable tubing. There can be multiple sizes of masks, e.g., adult, child and infant. There can be an elastic strap that holds the mask in the correct placement over the patient's nose and mouth, the mask not being airtight on the patient's face. The tubing, which can be approximately 2 m long and have a funnel-shaped connector at the proximal end to ensure a simple and safe connection to the oxygen source, can also be disposable and sterile, i.e., ready for use upon opening the a package containing the mask apparatus.

The quantity of oxygen delivered from a storage tank to the mask is controlled by a value regulator on the oxygen tank or source. The final oxygen concentration delivered by a simple face mask depends upon the amount of ambient air that mixes with the oxygen that the patient breathes. The air mixing is determined by how much air the patient is breathing at the time in light of the extent the mask fits on the patient's face. Due to the variability in these factors, the final oxygen concentration can be relatively uncontrolled. Use of a mask allows delivery of between 6 to 10 liters of gas per minute. Such a mask is meant for patients who are able to respire on their own, but who may require a higher oxygen concentration than the 21% concentration found in ambient air.

The mask can include a coating or embedded material on the inside portion of the mask that rests adjacent to the patient's face where the patient exhales, such as adjacent the patient's nose or mouth. The coating or embedded material serves as a visual indicator and changes color and/or becomes more or less opaque or translucent upon exposure to the patient's exhaled breath, returning to its original color, e.g. translucent, upon inhalation or when the patient is at rest or stops respiring, i.e., is not inhaling or exhaling. The coating or embedded material includes a substance that reacts to one or more components of the patient's exhaled breath, e.g., water vapor, change in temperature, or exhaled CO₂. The color change can occur with each breath that the patient takes, which may be from 1 to 40 times per minute. The color changes can be between translucent and a partially opaque or opaque color, or between two different partially or fully opaque colors, such as red, green, or blue. For example, using a clear mask and a coating that reacts to the water vapor in exhaled air during exhalation only, the mask would be colored and/or opaque when the patient is not exhaling, and clear when the patient is exhaling. The change of color provides a visual color clue that the patient is not obstructing and is continually exchanging air in a rhythmic pattern at a time when the patient may be at risk or airway obstruction, e.g., during immediate post-operative post-anesthesia recovery, during transport into the post anesthesia care unit (PACU), and during recovery in the PACU. The mask can also be used intra-operatively during monitored anesthesia care (MAC) cases, in concert with or in lieu of an end tidal CO₂ measurement. The mask can also be used in the intensive care unit (ICU) and non medical-surgical floors. A principal purpose of the mask is to have a visual cue that the patient is actively respiring with air exchange and not obstructing during immediate post-operative recovery, post-anesthesia and intra-operative (MAC cases and regional anesthesia cases) and/or while the patient needs or receives oxygen via a face mask. In general, the mask should only be used when the patient is in need of oxygen via a face mask or is at risk for obstructing or apnea.

The coating or embedded material can be any material, such as hydrochromic coatings or resins or thermochromatic resins or inks, which can be a leuco dye, that creates reversible color-change upon respiration. In one embodiment, the material can be a thermochromatic resin sold under the trade name CHROMICOLOR® (Matsui International Co. Inc., Gardena, Calif.). The coating or embedded material can be a silica gel, which is used as a desiccant that changes color with exhalation. The coating or embedded material can include a mixture of at least one basic or acid color former or dye, preferably a triphenyl methane system, and at least one complementary acid or basic compound, which mixture reversibly changes color in response to a gas, vapor or temperature change. The coating or embedded material can include a CO₂ indicator composition, which can be bromthymol blue that changes from, for example, blue to yellow when exposed to carbon dioxide, a pH indicator and a long-lived reference luminophor and/or a porous sol-gel matrix, or a hydrophilic, gas permeable, crosslinked polyurethane.

More specifically, the visual indicator can be a thermochromatic chromicolor molding (e.g., a color changing plastic), a thermochromatic chromicolor ink, and/or a hydrochromic ink. In some embodiments, the visual indicator is a thermochromatic leuco dye (TLD) that changes from a first color state (e.g., a visible color) at lower temperature to a second color state (e.g., translucent) when at a higher temperature. The color change is reversible, and the dye returns to its first color state when the temperature of the mask and/or dye decreases a few degrees Celsius below the exhalation temperature.

In some embodiments, the mask has a thermochromatic visual indicator, such as a large green circle or “X”, which serves as an indicator that a patient is or is not breathing. The visual indicator disappears at elevated temperature, for example as the patient is exhaling, and reappears when the temperature decreases, for example when the patient is inhaling, obstructing, or not breathing. In some embodiments, the default color state of the visual indicator is clear, and the visual indicator turns a visible color (e.g., green) when the patient exhales into the mask. If the patient is receiving oxygen or other gas through the mask, the temperature of and humidity in the mask drop as each exhalation is cleared from the mask. Thus, the visual indicator can be calibrated to transition between each breath and/or to transition after a predetermined period of time, such as if the patient stops breathing for several seconds.

The visual indicator can be configured in any suitable shape, size, color, location, or opacity. By way of non-limiting example, the visual indicator can be a circle, square, triangle, or “O”; the visual indicator can be applied to substantially all of the mask, to a portion of the mask, and/or to one or more specific locations on the mask; and/or the visual indicator can be opaque, translucent, or transparent.

One of skill in the art can formulate TLDs to change color at temperatures ranging from about −15 to about +60 degrees Celsius (about 5 to about 140 degrees Fahrenheit) and to have a specific sensitivity. For example, TLDs can be formulated to transition in response to a relatively small temperature change, such as, for example, from about 3 to about 10 degrees Celsius (about 5 degrees to about 18 degrees Fahrenheit).

Preferably, the visual indicator is composed of a non-toxic material that can be embedded in the mask or coated on a surface of the mask. Beneficially, the visual indicator can be applied as part of a label, such as a sticker or decal, that can be placed on or inside a respiratory mask. In some embodiments, the label can be clear so as not to interfere with the visibility of the visual indicator. The label can include an adhesive for affixing the label to a mask. The label can have as a backing or substrate any suitable material, such as sheet plastic, PVC, or fabric. The visual indicator can be printed, coated, painted, impregnated, extruded, or applied on one or more surfaces of the label backing or mask. In preferred embodiments, the visual indicator would be in a first color state (e.g., clear) at ambient temperatures, such as the temperature of an operating room (e.g., about 65 degrees to about 75 degrees Fahrenheit), and the visual indicator would transition to the second color state (e.g., a solid color) when exposed to the temperature of an exhaled breath (e.g., about 89 to about 101 degrees Fahrenheit).

An exemplary color changing material is CHROMICOLOR® (Matsui International Color; Garden, Calif.), which is available as a temperature changing resin and as a temperature changing ink. To manufacture a mask with an embedded visual indicator, the process begins with CHROMICOLOR® resin concentrates which are combined with the fabrication material, such as clear plastic. The mixture is then cast or molded into a mask that possess temperature-sensitive, color-changing properties. In some embodiments, temperature changing ink can be applied on an interior surface or an exterior surface of the mask, or as part of a label that is affixed to the mask.

Another color changing material is HYDRO-CHROMIC WHITE™ (Matsui International Color; Garden, Calif.), which is a binder that repeatedly changes from white (or colored) to transparent when contacted with water. The binder returns to white (or colored) as it dries. Therefore, the binder can be used to conceal a design on the mask, such as a red “X”. The binder can be compounded with colored pigments to aid in concealing a design or symbol, and to give the binder a translucent color when wetted. As a patient exhales, condensation from each breath forms on the inside of the mask, which causes the binder to turn translucent to reveal the design. When a patient is inhaling, between breaths, or not having respiratory exchange, the condensation is at least partially dissipated by the flow of gas though the mask, and the binder returns to its opaque white or colored state. Thus, a reversible visual cue is created for the patient care provider. Preferably, this visual indicator is located on an interior surface of the mask where it will come into contact with condensation from the patient's breath. The visual indicator also can be applied as part of a label that can be placed inside a mask. For example, the label can incorporate the design and/or the color changing binder. The label can include an adhesive for affixing the label to the mask.

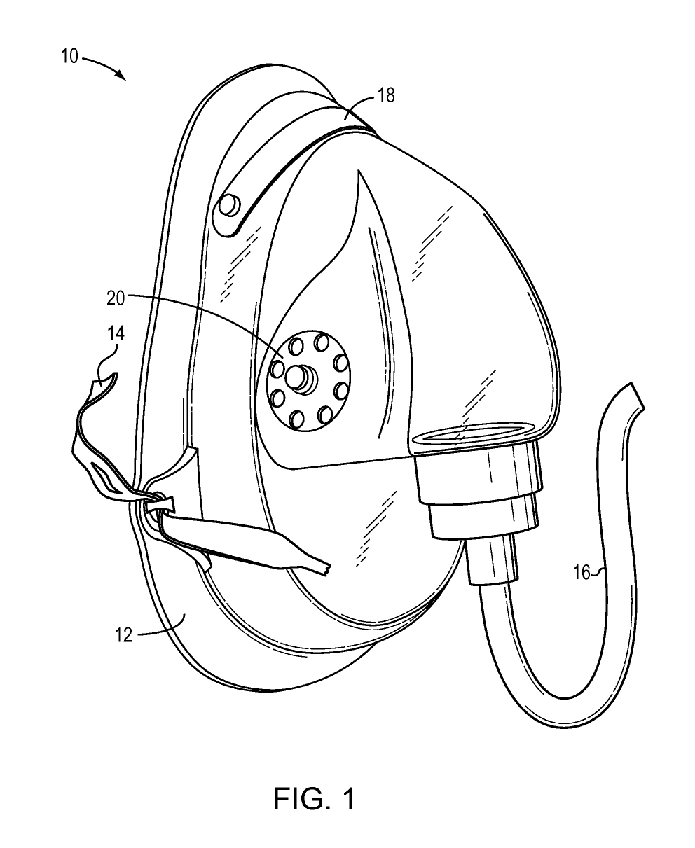

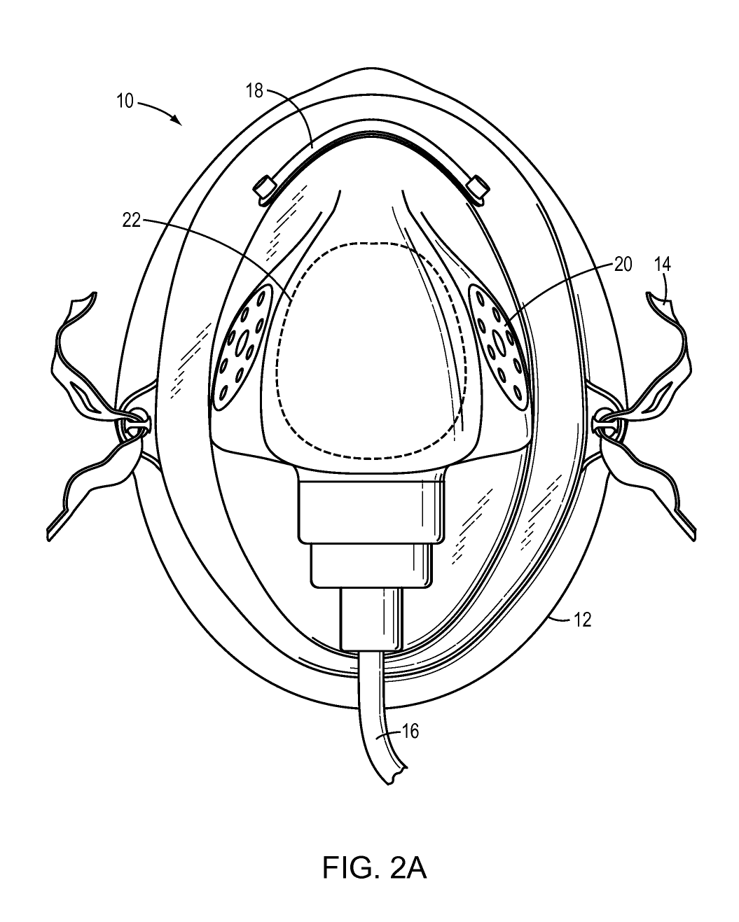

FIG. 1 shows a perspective view of a respiration mask 10. Mask 10 includes a body 12 configured to cover a patient's nose and mouth and an adjustable or elastic strap 14 for securing mask 10 to the patient's face. Tube 16 connects mask 10 to a gas source. Mask 10 optionally includes a nose clip 18 for conforming the mask to the patient's nose. In addition, mask 10 can include one or more holes or perforations 20 that allow air to enter and exit the mask. The holes can be adjustable to allow more or less air to pass through.

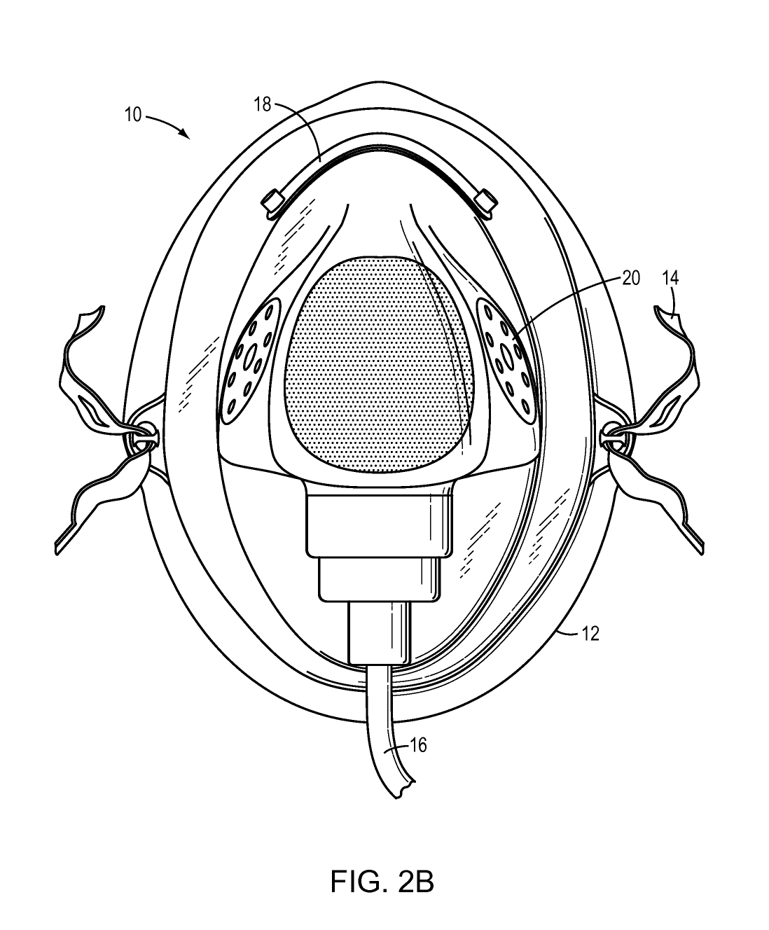

FIG. 2A shows a top view of a respiration mask having a circle-shaped visual indicator 22 denoted by the dotted line. The visual indicator changes from a clear color state (FIG. 2A) when the patient is between breaths or stops breathing, to a visible color state (e.g., bright green) when the patient exhales into the mask (see FIG. 2B). In some embodiments, the visual indicator repeatedly appears each time the patient exhales and disappears each time the patient inhales or stops breathing.

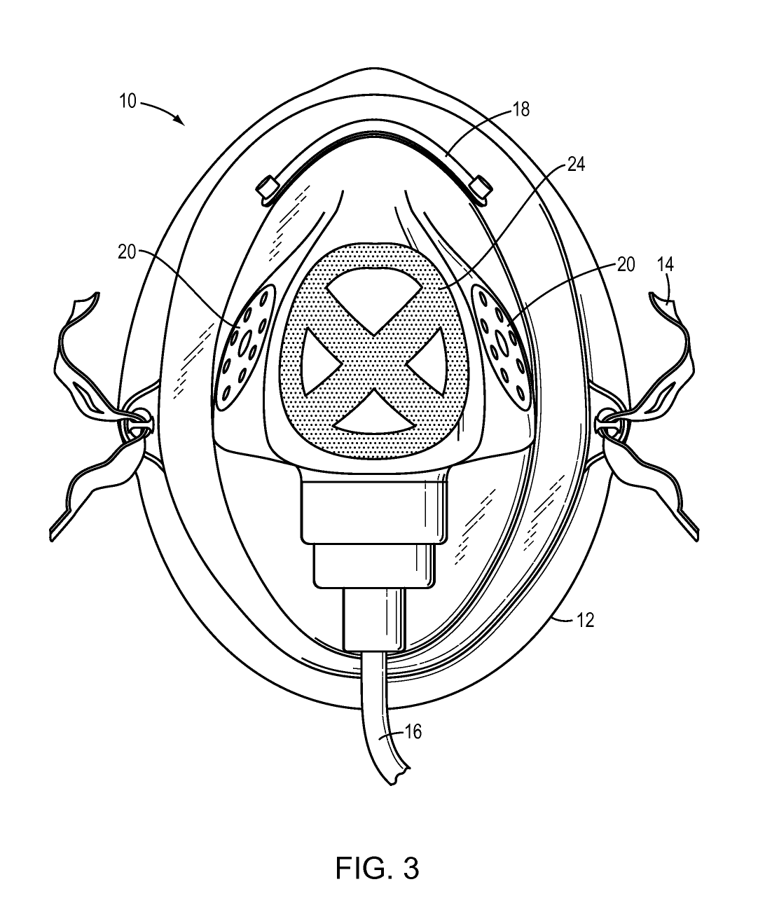

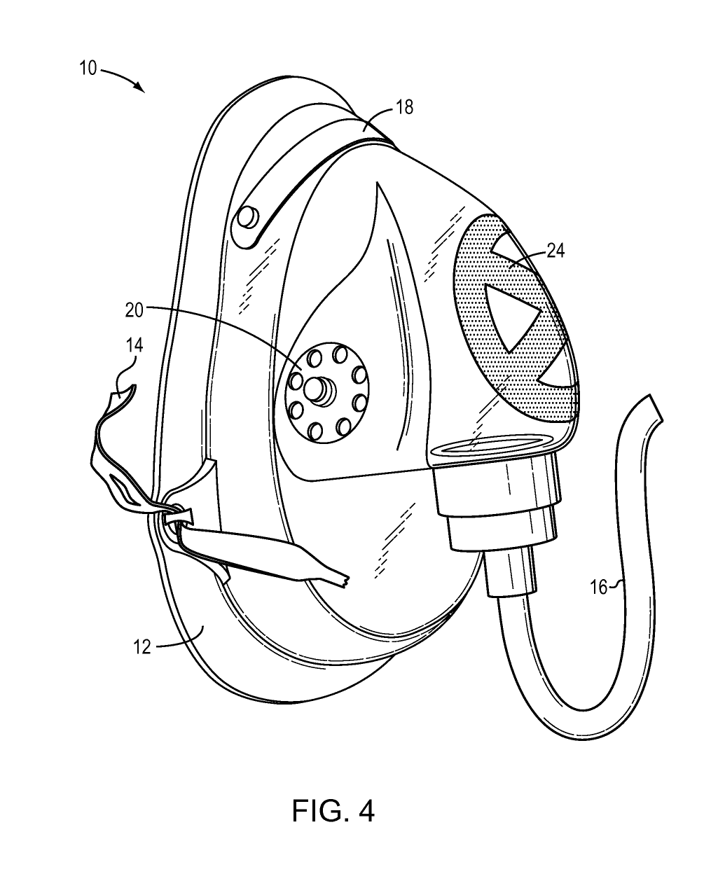

FIG. 3 shows a top view of a respiration mask 10 where the visual indicator 24 is an “X” in a circle. FIG. 4 shows a perspective view of the mask shown in FIG. 3. In FIG. 3, the visual indicator changes from a visible color state (e.g., a red “X”) when the patient is between breaths or is not having respiratory exchange (FIG. 3), to a clear color state when the patient exhales into the mask (see FIG. 1). In some embodiments, the visual indicator repeatedly appears each time the patient exhales and disappears each time the patient inhales or stops having respiratory exchange.

Methods are well known for applying color changing inks and for preparing plastics containing color changing resins. See, e.g., McGrawHill Yearbook of Science and Technology 2008; Bamfield, Chronic Phenomena: Technological Applications of Colour Chemistry, 2001; and Muthyula, Chemistry and Applications of Leuco Dyes, 1997. For example, thermochromatic inks can be microencapsulated using an interfacial polymerization process. Suitable microencapsulated dyes include those having an average diameter of about 2 to about 5 microns. Briefly, an internal phase (i.e., the material inside the microcapsule), an external phase (e.g., a wall material of the microcapsule), and water are combined under homogenization to make a stable emulsion of the desired particle size, usual 5 microns or below. By controlling process conditions precisely, such as temperature, pH, reagent concentrations, mixing speed, etc., the external phase will surround internal phase droplets and crosslink to form microcapsules. Once microencapsulated, color changing dyes can be incorporated into a mask.

In the description, the invention is discussed in the context of respiration masks in the context of a hospital or care provider setting; however, these embodiments are not intended to be limiting and those skilled in the art will appreciate that mask also can be used in other settings and in other configurations.

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention.

What is claimed is:

1. A respiratory mask comprising:
a body forming a respiratory mask shaped to enclose a patient's nose and mouth, the body configured to deliver breathable gases to the patient; and
a visual indicator associated with the body, the visual indicator being capable of transitioning from a first color state to a second color state in response to a patient's respiratory exchange.

2. The respiratory mask of claim 1, wherein the visual indicator is a color changing dye.

3. The respiratory mask of claim 1, wherein the visual indicator transitions from the first color state to the second color state when the patient exhales.

4. The respiratory mask of claim 1, wherein the visual indicator is capable of transitioning from the second color state to the first color state between exhalations.

5. The respiratory mask of claim 1, wherein the visual indicator is responsive to temperature changes.

6. The respiratory mask of claim 5, wherein the visual indicator transitions from the first color state to the second color state when the visual indicator reaches a temperature of about 89 degrees Fahrenheit.

7. The respiratory mask of claim 1, wherein the visual indicator is responsive to moisture contained in breath.

8. The respiratory mask of claim 1, wherein the visual indicator is responsive to CO₂ levels contained in breath.

9. The respiratory mask of claim 1, wherein the first color state is selected from the group consisting of: clear, a visible color, opaque, translucent, and/or combinations thereof.

10. The respiratory mask of claim 1, wherein the second color state is selected from the group consisting of: clear, a visible color, opaque, translucent, and/or combinations thereof.

11. An indicator for monitoring patient respiratory exchange comprising: a backing and a visual indicator disposed on the backing, the visual indicator being capable of transitioning from a first color state to a second color state in response to a patient breath, wherein the indicator is adapted to be affixed to a respiratory mask.

12. The indicator of claim 11, wherein the visual indicator is a color changing dye.

13. The indicator of claim 11, wherein the visual indicator transitions from the first color state to the second color state when the patient exhales.

14. The respiratory mask of claim 11, wherein the visual indicator transitions from the second color state to the first color state between exhalations.

15. The indicator of claim 11, wherein the visual indicator is responsive to temperature changes.

16. The indicator of claim 15, wherein the visual indicator transitions from the first color state to the second color state when the visual indicator reaches a temperature of about 89 degrees Fahrenheit.

17. The indicator of claim 11, wherein the visual indicator is responsive to moisture contained in breath.

18. The indicator of claim 11, wherein the visual indicator is responsive to CO₂ levels contained in breath.

19. The indicator of claim 11, comprising an adhesive disposed on a first side of the backing, the adhesive capable of affixing the indicator to a respiratory mask.

20. The indicator of claim 11, wherein the first color state is selected from the group consisting of: clear, a visible color, opaque, translucent, and/or combinations thereof.

21. The indicator of claim 11, wherein the second color state is selected from the group consisting of: clear, a visible color, opaque, translucent, and/or combinations thereof.

22. A system for monitoring patient respiratory exchange, the system comprising:
a respiratory mask shaped to cover a patient's nose and mouth and configured to deliver breathable gases to the patient;
a visual indicator disposed on the respiratory mask, the visual indicator being capable of transitioning from a first color state to a second color state in response to a patient's respiratory exchange; and
an electronic monitor for monitoring the status of the visual indicator, the electronic monitor configured to trigger an alarm if the visual indicator remains in the first color state for a period of time substantially longer than the duration of a single breath.

23. A method of monitoring patient respiratory exchange, the method comprising:
providing a respiratory mask having a visual indicator disposed on the respiratory mask, the visual indicator being capable of transitioning from a first color state to a second color state in response to a patient's respiratory exchange;
electronically monitoring the visual indicator, wherein rhythmic transitions between the first color state and the second color state are indicative that the patient is exchanging normally; and
electronically triggering an alarm if the visual indicator remains in the first color state for a period of time substantially longer than the duration of a single breath.
